(19) United States
(12) Patent Application Publication
(10) Pub. No.: US 2004/0111077 A1
(43) Pub. Date: Jun. 10, 2004

(54) Safes

(76) Inventor: Exie Smith, St. Louis, MO (US)

Correspondence Address: Exie Smith, Apt-3, 1217 Covington MN-LN, St. Louis, MO, 63125, US

(21) Appl. No.: 10/314,468

(22) Filed: Dec. 9, 2002

(51) Int. Cl. A61F 13/15; A61F 13/20

(52) U.S. Cl. 604/385.17

(57) ABSTRACT

What is new about safes the invention I want patented is, no one has ever put the kotex with wings? and a tampon togehter as one product!

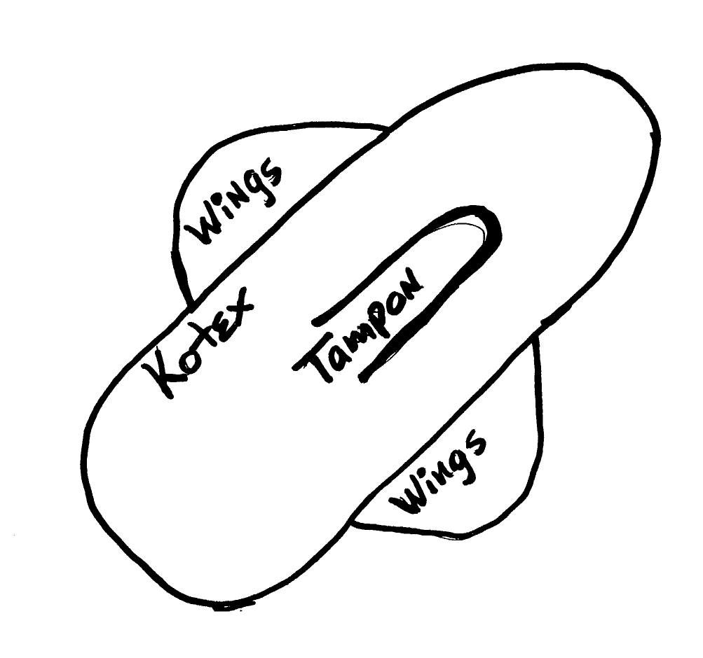

and for those women whom get sore during this time of the month?

There will be a package of hydrocortisone 15 ointment to help heal the sore area!

Safes are a conbiunation of kotex and tampon put togehter to form one, with wings on each side.

To make the safes product, I would glue a tampon to the middle of a kotex with wings and that would be? the whole invention.

to use this invention, you fold back the ends of the kotex so to incert the tampon. Into the vagina, then spread out the kotex and affix the wings to each side of the seat of the panties.

1. Is a one and only product that has never been marketed? the way I have put them togehter to form one product as a whole to stop most leakage? if not all leakage of women menstral period!

2. This product has 4 different ways to stop and help to heal the soreness that? Some women have on their menstral period!
a tampon to stop most of fluids.
a kotex to catch any leakage.
wings to hold everything in place.
hydrocortisone 15 ointment for to help heal!

3. And this ointment can help to heal women, whom just had their babies!
